(19) United States
(12) Patent Application Publication
(10) Pub. No.: US 2005/0084537 A1
(43) Pub. Date: Apr. 21, 2005

(54) Sustained-release compositions

(76) Inventors: Glen Patrick Martyn, Nottingham (GB); Julian Blair, Nottingham (GB)

Correspondence Address: Saliwanchik Lloyd & Saliwanchik, 2421 N W 41st Street, Suite A-1, Gainesville, FL, 32606-6669, US

(21) Appl. No.: 10/496,208

(22) PCT Filed: Dec. 9, 2002
(86) PCT No.: PCT/GB02/05563

(30) Foreign Application Priority Data
Dec. 10, 2001 (GB) 0129489.1

(51) Int. Cl. A61K 9/14

(52) U.S. Cl. 424/489

(57) ABSTRACT

Microparticles comprise a therapeutic agent dispersed within a polymer matrix, the matrix comprising a first polymer of hyaluronic acid and a second polymer of either a non-ionic polymer, a polymeric gum or a combination thereof. The microparticles may be formulated for nasal or pulmonary delivery.

### FIELD OF THE INVENTION

The present invention relates to sustained-release compositions of a therapeutic agent incorporated in solid microparticles, and methods for their production.

### BACKGROUND OF THE INVENTION

The two main advantages of using controlled release products in the pharmaceutical field are the ability to maintain an elevated therapeutic plasma level over a prolonged period of time and an increase in patient compliance obtained by reducing the number of doses necessary to achieve the same effect with a rapid-acting formulation.

Many controlled release delivery systems are commercially available. For example, both oral and transdermal formulations are well known in the art. Oral inhalation therapy is used commonly for the delivery of drugs in the treatment of asthma, cystic fibrosis, etc. There are many different delivery devices which may be used to administer drugs to a patient via the inhalation route, such as nebulizers, pressurized metered dose inhalers (PMDIs) and dry powder inhalers (DPIs).

Many of the currently available inhalation systems present the active in a rapid-acting format although some drugs, such as salmeterol, have been chemically modified to provide a sustained bronchodilatory effect. Increasing attention is now being given in the art to the use of this route of administration for the delivery of peptides and proteins as these are usually administered by injection because of their poor absorption via oral administration. Furthermore, it is desirable to avoid repeated injections.

Numerous studies have been conducted to develop biodegradable polymers suitable for use in sustained-release drug formulations. Biodegradable polyesters such as polylactide, polyglycolide, poly(lactide-co-glycolide), poly-ortho-ester and polyanhydride have been found to be effective in such use (Chasin et al., Biodegradable Polymers as Drug Delivery Systems, 1990; MercelDekker, and Heller, Ady. Drug Del. Rev., 1993; 10: 163). Other studies using natural polymer materials such as gelatin, collagen, chitosan, carboxymethyl cellulose, alginate and hyaluronic acid have also been published.

Meyer et al., J. Controlled Release, 1995; 35: 67, reported a sustained-release formulation of G-CSF employing a gel containing 0.5 to 4% hyaluronic acid. However, administration was by injection in the gel state. Japanese Patent Publication No. 1-28704](1989) discloses that when a sustained-release injection formulation of insulin containing 1% hyaluronic acid is administered to rabbits, the therapeutic effect of suppressing the blood glucose level does not last more than 24 hours. Accordingly, a sustained release drug formulation based on hyaluronic acid gels has the disadvantage that the drug release cannot be maintained for more than 24 hours and is difficult to inject due to the high intrinsic viscosity of the gel. Such formulations are also unsuitable for pulmonary delivery.

Natural hyaluronic acid, and inorganic salts thereof, dissolve only in water. However, drug compositions comprising solid microparticles of hydrophobic hyaluronic acid derivatives have been prepared by the conventional emulsion-solvent extraction method (Nightlinger et al., Proceed. Intern. Control. Rel. Bioact. Mater., 1995; 22nd: Paper No. 3205; Ilum, et al., J. Controlled Rel., 1994; 29: 133). However, this method results in the denaturation of the protein active through its contact with an organic solvent.

Thus, a sustained-release formulation which does not require the repeated daily administrations of a therapeutic protein is highly desirable. The delivery system should also present the active ingredient in a relatively bioavailable form.

### SUMMARY OF THE INVENTION

The present invention is based on the surprising finding that a sustained release composition can be prepared using the polymer hyaluronic acid, or a derivative thereof, and a further polymer which may be a non-ionic polymer or a hetero or homo polymeric gum.

According to a first aspect of the invention, a microparticle comprises a therapeutic agent dispersed within a polymer matrix, the polymer matrix comprising a first polymer of hyaluronic acid, or a derivative thereof, and a biodegradable second polymer.

According to a second aspect of the invention, a method for the production of microparticles suitable for pulmonary administration comprises the steps of:
a) mixing a therapeutic active with a hyaluronic acid polymer, or salt thereof, to form an aqueous gel;
b) adding the gel to a stirred non-aqueous solvent to form a dispersion of gel microdroplets; and
c) drying the dispersion to form dried microparticles.

This method allows concentrated solutions of hyaluronic acid and therapeutic active to be prepared in a form that allows suitable drying methods, for example, spray-drying, to be used.

According to a third aspect of the invention, a method for the production of microparticles suitable for pulmonary administration, comprises mixing divalent metal cations with an aqueous solution or suspension comprising a therapeutic agent and a hyaluronic acid polymer, or salt thereof, and processing the resulting product to form microparticles.

This method results in microparticles having a prolonged release profile.

The microparticles of the invention can be used to deliver a wide variety of medicaments and provide a reproducible in vivo therapeutic effect when a desired unit dose of the microparticles are administered to a patient.

### DESCRIPTION OF THE INVENTION

The invention is described with reference to the accompanying drawings, wherein:

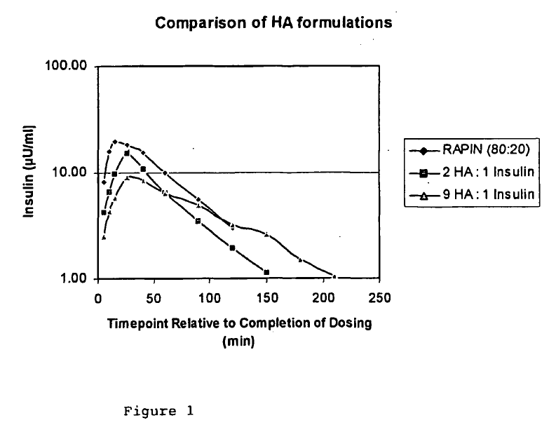

FIG. 1 is a graphic representation of the release of insulin from microparticles formulated with hyaluronic acid only; and

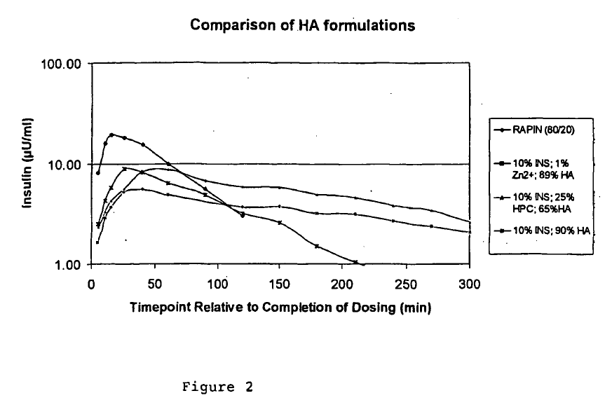

FIG. 2 is a graphic representation of the release of insulin from microparticles of the invention.

### DESCRIPTION OF THE INVENTION

The microparticles according to the invention may be adapted for any suitable route of administration, i.e. oral ocular, rectal, vaginal etc. For example, a composition comprising the microparticles may be prepared for delivery via injection, including subcutaneous injection, transdermal injection, intramuscular injection or using ballistic/needle-free injection systems. In a preferred embodiment, the microparticles are to be delivered via inhalation, i.e. nasal or pulmonary administration.

The microparticles may also be prepared for delivery as tablets, in capsules, pessaries, suppositories, etc.

The microparticles of the invention provide controlled release of a therapeutically active agent dispersed in the polymer matrix. For the purposes of the present invention, the term “controlled release” means that the therapeutically active agent is released from the microparticle at a controlled rate such that a therapeutically beneficial amount of the material is delivered to a patient over an extended period of time, e.g. providing a dosage form which provides effective levels of medicament in vivo for a time period of from about 1 to about 24 hours, or more.

The microparticles are generally of a size less than 100 μm in diameter, depending on the route of administration. For delivery via oral or nasal inhalation, the microparticles will typically be of a size of about 0.1 μm to 50 μm in diameter. Preferably, the microparticles are of a size of about 0.1 μm to 5 μm in diameter. More preferably, the microparticles will be of a size of about 2 μm in diameter in order for the microparticles, when inhaled, to reach the alveoli of the lungs.

In the first aspect of the invention, the microparticles comprise a polymer matrix formed from a first polymer of hyaluronic acid and a biodegradable second polymer.

As used herein, the reference to “a polymer matrix” is intended to mean that the polymers used in the composition form a stable structure capable of retaining the therapeutic agent that is dispersed therein. Polymers are typically made up of multiple repeating monomer units, typically greater than 3 monomer units.

Hyaluronic acid is a commercially available polymer and which exhibits good mucoadhesive properties, is biocompatible, biodegradable and is also able to avoid phagocytic uptake. Derivatives of hyaluronic acid are known and can be formed via esterification (both internal and external), cross-linking (e.g. via photo- or glutaraldehyde), or sulphation. Suitable commercial materials include HYAFF (Fidia), ACP (Fidia), Intergel (LifeCore), Incert (Anika) and Hylans (BioMatrix). Particularly preferred derivatives are those obtained by coupling or conjugation, such as SepraFilm (Genzyme) which is a conjugate of carboxymethylcellulose and hyaluronic acid. With this latter material, there is no need to include the second polymer, although that may be an optional step.

The second polymer is a biodegradable polymer different from the hyaluronic acid polymer. Suitable polymers include ionic or non-ionic polymers, including cellulose or cellulose derivatives. Preferred examples include carboxymethyl cellulose, hyroxypropylmethyl cellulose (HPMC), hydroxyethyl cellulose, hydroxypropyl cellulose, or mixtures thereof. Non-ionic polymers are preferred. The second polymer will preferably have a molecular weight greater than 100 kDa, most preferably greater than 500 kDa. The second polymer provides a synergistic gelling effect with the anionic hyaluronic acid.

In a further separate embodiment, the biodegradable second polymer may be a natural heteropolymeric or homopolymeric gum, or a combination thereof, to provide a similar synergistic effect. The term “heteropolymer” as used in this embodiment is used to define a water-soluble polysaccharide containing two or more different sugar sub-units. The heteropolymer may have a branched or helical configuration. In a preferred embodiment, the heteropolymer has a molecular weight greater than 500 kDa. An especially preferred heteropolymer is xanthan gum, which is a high molecular weight (approximately 1,000 kDa) heteropolysaccharide.

The homopolymers useful in the invention include galactomannan gums. Locust bean gum, which has a higher ratio of mannose to the galactose, is especially preferred as compared to other galactomannans such as guar and hydroxypropyl guar. Other naturally occurring polysaccharide gums known to those skilled in the food and pharmaceutical arts are also useful in combination with the hyaluronic acid polymer to provide an improved controlled release carrier of the invention. These gums include alginic acid derivatives, carageenans, tragacanth, acacia, karaya, the polyethylene glycol esters of these gums, chitin, chitosan, mucopolysaccharides, konjac, starch, substituted starches, starch fragments and dextrins. The homopolymer will preferably have a molecular weight greater than 100 kDa, most preferably greater than 500 kDa.

The relative amounts of first and second polymers in each microparticle can be varied depending on the desired release characteristics. The hyaluronic acid polymer will usually be in excess of the second polymer. Typically, the hyaluronic acid will be present in an amount of 0.1% to 99% by weight of the composition, preferably 10% to 80% by weight, most preferably 25% to 75% by weight.

In a preferred embodiment (when a non-ionic polymer is used), a cationic cross-linking agent may be included in the microparticles of the invention. The cationic cross-linking agent may comprise, e.g. monovalent or multivalent metal cations. Specific examples of suitable cationic cross-linking agents include calcium chloride, sodium chloride, potassium chloride, potassium sulfate, sodium carbonate, lithium chloride, tripotassium phosphate, sodium borate, potassium bromide, potassium fluoride, sodium bicarbonate, magnesium chloride, sodium citrate, sodium acetate, calcium lactate, and sodium fluoride. Multivalent metal cations may also be utilized. However, the preferred cationic cross-linking agents are monovalent or divalent. Particularly preferred salts are potassium chloride, calcium chloride and sodium chloride. The cationic cross-linking agent is included in the controlled release inhalation formulations of the present invention in an amount from 0.01 to 50% by weight, preferably from 1% to 20% by weight, more preferably from 0.1% to 10% by weight of the non-hyaluronic acid polysaccharide component.

In a second aspect of the invention, the microparticles are prepared by mixing a therapeutic active with a hyaluronic acid polymer to form an aqueous gel, and subsequently adding the gel to a stirred non-aqueous solvent to form a dispersion, which can then be dried. In this aspect, there is no need to include the second polymer, although that may be an optional step. If a second polymer is to be included, the polymer will be added during the step of forming the gel.

In preferred embodiments, the solvent is a perfluorocarbon, e.g. perfluorodecalin or perfluoro-n-octane. These materials are non-reactive, thereby preserving the bioactivity of the therapeutic agent. The solvents also have a low vapour pressure and so can be atomised readily.

In a third aspect, the microparticles are prepared using a divalent metal cation, without the additional requirement for the second polymer (although this is optional). Suitable divalent metal cations are referred to above, including zinc, lithium, calcium, ammonium, magnesium, copper and cobalt salts. In this aspect, the therapeutic agent, hyaluronic acid polymer and divalent metal cations are added together to form an aqueous solution or suspension and then processed to form microparticles.

Any suitable therapeutic agent may be used in the present invention, as will be appreciated by the skilled person. Therapeutic agents which may be used include, for example, proteins, peptides, nucleic acids and small organic molecules. Anti-inflammatory compounds are preferred, as is insulin in its hexameric or monomeric form. The reference to therapeutic agents is intended to also include prophylactic agents, including vaccines in the form of proteins or polypeptides, attenuated and live microorganisms or viruses. Suitable adjuvants may also be incorporated into such a vaccine composition. Pharmaceutical agents that are particularly suitable for administration via the pulmonary route are preferred, in particular, antiallergics, bronchodilators, analgesics, antibiotics, antihistamines, anti-inflammatories, steroids, cytokines, cardiovascular agents and immunoactive agents.

Particularly preferred therapeutic agents include: human growth hormone, bovine somatotropin, porcine somatotropin, growth-hormone-releasing peptide, granulocyte-colony stimulating factor, granulocyte macrophage-colony stimulating factor, macrophage-colony stimulating factor, erythropoietin, bone morphogenetic protein, interferon, insulin (which includes human insulin and chemically modified forms of insulin, insulin lispro, insulin porcine, insulin NPH, protamine-insulin, insulin aspart, insulin glargine and insulin detemir), atriopeptin-III, monoclonal antibody, TNF, macrophage-activating factor, interleukin, tumor-denaturing factor, insulin-like growth factor, epidermal growth factor, tissue plasminogen activator and urokinase.

The therapeutic agent is “dispersed” within the polymer matrix. This term is used herein to refer to the retention of the therapeutic agent within the polymer. Typically, the therapeutic agent will be dispersed uniformly throughout the matrix, although this is not necessarily required for the practice of the invention.

It will be appreciated by the skilled person that the therapeutic agents are to be formulated in physiologically effective amounts. That is, when delivered in a unit dosage form, there should be a sufficient amount of the therapeutic agent to achieve the desired response.

If the microparticles are intended for delivery as dry powders in an inhalation device, it will be appreciated that a unit dose comprises a predefined amount of microparticles delivered to the patient in one inspiratory effort. In a preferred embodiment, the microparticles are prepared as single unit dosage forms for inclusion in dry powder inhalers. In this embodiment, a single unit dose will be approximately 1 to 15 mg, preferably between 5 to 10 mg.

The amount of therapeutic agent present in each microparticle will be determined on the basis of the level of biological activity exhibited by the therapeutic agent. If the therapeutic agent has high activity, then there may be as little as 0.001% w/w of the agent. with respect to the polymer material. Usually the microparticles will comprise greater than 5%, 20%, 30% or even 40% w/w of the therapeutic agent. The amounts can be controlled by regulating the concentration of the agent in solution with the polymer prior to forming the microparticles.

The composition delivered to a patient may also comprise other components, e.g. carbohydrates or other glass-forming substances as stabilisers or excipients. Additional components may be desirable to modify the characteristics of the microparticles. For example, it may be desirable to add further components to improve the particle rigidity or release profile.

The microparticles are intended primarily for delivery via pulmonary or nasal inhalation. The preferred delivery system is a dry powder inhaler (DPI), which may rely on the patient's inspiratory efforts to introduce the microparticles in a dry powder form into the lungs. However, alternative inhalation devices may also be used. For example, the microparticles may be formulated for delivery using a metered dose inhaler (MDI), which usually requires a high vapour pressure propellant to force the microparticles into the respiratory tract. Nebulisers are also envisaged. These require aerosol formulations, which will be apparent to the skilled person. Suitable excipients will be apparent to the skilled person. For pulmonary or nasal administration, suitable excipients include buffers, surfactants, density modifiers, penetration enhancers, etc.

In the context of dry powder inhalers, the microparticles may be formulated in compositions further comprising bulk carrier particles, which aid delivery. Suitable carrier particles are known, and include crystalline lactose particles and mannitol particles, of a diameter size typically in the range of from 30 to 300 μm, more usually 50 to 250 μm.

The microparticles may also be delivered via other suitable routes. In one embodiment, transdermal delivery may be used. In this context, the microparticles may be formulated with a suitable excipient, including non-aqueous vehicles, density modifiers, adjuvants, etc.

It may be desirable to add a pharmaceutically acceptable surfactant to the compositions of the invention in a sufficient amount to either modify release-controlling characteristics or the solubility characteristics of the therapeutic agent. In such embodiments, the surfactant comprises from about 0.01 to about 10% of the controlled release carrier, by weight, and more preferably from about 0.1 to about 2% of the controlled release carrier, by weight. The surfactants which may be used in the present invention generally include pharmaceutically acceptable anionic surfactants, cationic surfactants, amphoteric (amphipathic/amphiphilic) surfactants, and non-ionic surfactants. Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, and alkyl sulfates.

Suitable pharmaceutically acceptable non-ionic surfactants such as, for example, polyoxyethylene compounds, lecithin, ethoxylated alcohols, ethoxylated esters, ethoxylated amides, polyoxypropylene compounds, propoxylated alcohols, ethoxylated/propoxylated block polymers, and propoxylated esters, alkanolamides, amine oxides, fatty acid esters of polyhydric alcohols, ethylene glycol esters, diethylene glycol esters, propylene glycol esters, glyceryl esters, polyglyceryl fatty acid esters, SPAN's (e.g. sorbitan esters), TWEEN's, sucrose esters, and glucose (dextrose) esters. The surfactant should be non-sternutatory so as not to irritate the mucous membrane.

Other suitable pharmaceutically acceptable surfactants/co-solvents (solubilizing) agents include acacia, benzalkonium chloride, cholesterol, emulsifying wax, docusate sodium, glyceryl monostearate, lanolin alcohols, lecithin, poloxamer, poloxyethylene castor oil derivatives, poloxyethylene sorbitan fatty acid esters, poloxyethylene stearates, sodium lauryl sulfates, sorbitan esters, stearic acid, and triethanolamine.

Mixed surfactant/wetting agent systems are also useful in conjunction with the present invention. Examples of such mixed systems include, for example, sodium lauryl sulfate/polyethylene glycol (PEG) 6000 and sodium lauryl sulfate/PEG 6000/stearic acid. The microparticles may also be prepared with suitable fillers, including sugars such as sucrose, dextrose, lactose, galactose, fructose, trehalose, mixtures thereof, as well as sugar alcohols such as mannitol, sorbitol, xylitol, lactitol, maltitol and galactitol. It is preferred that a soluble pharmaceutical filler such as lactose, dextrose, galactose, sucrose, or mixtures thereof be used. In addition, it is to be understood that the above-mentioned sugars and sugar alcohols can also be used as carriers as well, in place of or in addition to the materials described above.

Microparticles according to the invention may be prepared (processed) using any suitable technique, including spray-drying, vacuum drying, supercritical processing (e.g. SEDS, RESS, PCA), lyophilisation and milling procedures.

A particularly suitable technique is spray-freeze-drying technology. The process of spray-freeze-drying involves the atomisation of a solution or dispersion of the matrix-forming polymer(s) and therapeutic material, and then directing the resulting droplets into a liquified gas, typically liquid nitrogen, or a cryogenic surface. The droplets freeze on contact and may then be dried using a freeze-drying step to remove residual moisture. The resulting microparticles comprise a therapeutic agent dispersed within the polymer matrix.

Prior to microparticle formation, the therapeutic agent may be in solution or present as a dispersion of microparticles or nanoparticles (with an optional stabiliser) in the feedstock.

The apparatus and process conditions used to produce the initial droplets will be apparent to the skilled person. Feed concentrations, pump rates, atomisation pressures and nozzle types can all be selected based on conventional process conditions, and then optimised according to feedstock concentration and viscosity.

The size of the microparticles will be determined in part by the atomisation used in the spray-freeze-drying process. The atomisation/spraying stage may make use of a conventional atomisation process, e.g. pressure or two fluid nozzles, or may utilise an ultrasonic atomisation process (Maa et al., Pharmaceutical Research, 1999; 16(2)). The microparticles will usually have a mean aerodynamic particle diameter size ranging from 0.1 to 40 μm, preferably from 0.1 to 10 μm, and most preferably from 0.1 to 5 μm. This may be measured using an aerosizer as will be appreciated by the skilled person.

The drying process may be carried out using conventional freeze-drying apparatus. Drying will usually be carried out to achieve a residual moisture content of the microparticles of less than 10% by weight, preferably less than 5% by weight and most preferably less than 3% by weight.

The following Examples illustrate various aspects of the present invention. They are not to be construed to limit the claims.

### EXAMPLE 1

This is a comparative example, using only hyaluronic acid as the matrix former.

55.5 mg recombinant human insulin was dissolved in 0.77 ml 0.05M HCl with gentle agitation. To this was added 0.05 ml 1M NaOH dropwise, with gentle stirring. To this solution was added 155.6 ml of 0.3% w/v hyaluronic acid [2 MDa] and the solution was then made up to the desired volume with 17.58 ml purified water. 185 ml of this feedstock was then dried using an in-house spray-dryer under the following conditions; feed rate=2.0 g/min, inlet temp=130° C., outlet temp=89° C., atomisation=2-fluid nozzle, atomisation pressure=1.1 barg, atomisation flow rate=15 l/min, drying air pressure=1 barg, drying air flow rate=4.5 l/sec. Total recovery was low (11%). The final product had a mass ratio of 1:9 insulin:HA. A second formulation was prepared with a ratio of 1:2 insulin: HA. These formulations were then administered to beagle dogs, whose endogenous insulin was suppressed by i.v. administration of somatostatin. Each dry powder dose was delivered via a Penn-Century device through a tracheostome. The plasma insulin obtained can be seen in FIG. 1.

### EXAMPLE 2

A formulation containing 25% w/w HPC (ex. Nippo Soda Co., Japan) 10% w/w recombinant human insulin and 65% w/w high molecular weight hyaluronic acid was prepared as follows. To 154 mg insulin was added 2.16 ml 0.05M HCl and this was then swirled gently until the insulin dissolved. To this solution was added dropwise 0.14 ml 1M NaOH together with 165 ml purified water. This solution was then added to 96.25 ml of 0.4% w/v HPC solution and then 250 ml of a 0.4% w/v solution of high molecular weight hyaluronic acid was added and the mixture stirred until homogenous. Approximately 500 ml of this feedstock was spray dried at the following settings: feed rate=2.1 g/min, inlet temp=130° C., outlet temp=66° C., atomisation=2-fluid nozzle, atomisation pressure=2 barg, atomisation air flow rate=21 l/min, drying air pressure=1 barg, drying air flow rate=5 l/sec. Small powder doses were administered to dogs, as described in example 1.

The results are shown in FIG. 2. The profile demonstrates the increased duration of release observed with the composition obtained according to this invention when compared to the similarly-loaded HA:insulin binary formulation of Example 1.

### EXAMPLE 3

This Example illustrates the third aspect of the invention.

Insulin (0.1 g) was dissolved in hydrochloric acid (0.05 M, 1.4 ml). Sodium hydroxide (1 M, 0.09 ml) was added dropwise to the solution, until the initially precipitated insulin had redissolved. The insulin solution was then diluted with 106.8 ml water (99.3 ml for Zn²⁺ containing solutions). All feedstocks were prepared to give a loading of 10% w/w insulin in the dried powder.

ZnCl₂ (0.01 g) was dissolved in water (10 ml). Sodium hyaluronate (0.89 g) was gently dissolved in water (222.5 ml). The zinc solution was added to the HA solution, closely followed by the insulin solution; the whole feedstock was stirred gently to release trapped air bubbles.

In all cases, a feedstock of 0.3% w/v containing 1 g of total solids was prepared. This allowed efficient atomisation of the solution. Significantly higher concentrations could not be used due to the high viscosity of HA.

The release profile of the formulation was compared against that containing 10% insulin and 90% HA. The results are shown in FIG. 2, where the HA/Zn/insulin formulation shows a longer release profile than the insulin/HA blend.

1. A microparticle comprising a therapeutic agent dispersed within a polymer matrix, the polymer matrix comprising a first polymer of hyaluronic acid, or a derivative thereof, and a second biodegradable polymer.

2. The microparticle according to claim 1, wherein the second polymer is a non-ionic polymer, a polymeric gum, or a combination thereof.

3. The microparticle according to claim 1, wherein the therapeutic agent is a protein or peptide, the second polymer is a water-soluble polysaccharide containing two or more different sugar sub-units and the microparticle has a diameter less than 30 μm.

4. The microparticle according to claim 1, wherein the therapeutic agent is a protein or peptide, the second polymer is a homopolymeric gum and the microparticle has a diameter less than 30 μm.

5. The microparticle according to claim 1, wherein the second biodegradable polymer is cellulose or a cellulose derivative.

6. The microparticle according to claim 5, wherein the second polymer is carboxymethyl cellulose, hydroxypropylmethyl cellulose, hydroxyethyl cellulose, hydroxypropyl cellulose, or a mixture thereof.

7. The microparticle according to claim 1, further comprising a cross-linking agent.

8. The microparticle according to claim 1, wherein the therapeutic agent is a protein or peptide.

9. The microparticle according to claim 8, wherein the agent is insulin.

10. The microparticle according to claim 1, of a size from 0.1 to 40 μm in diameter.

11. The microparticle according to claim 10, of a size from 0.1 to 5 μm in diameter.

12. (canceled)

13. A composition comprising a microparticle comprising a therapeutic agent dispersed within a polymer matrix, the polymer matrix comprising a first polymer of hyaluronic acid, or a derivative thereof, and a second biodegradable polymer, wherein said composition is formulated for delivery via a means selected from the group consisting of oral or nasal inhalation, and transdermal delivery.

14. (canceled).

15. A composition comprising microparticles, the microparticles comprising a therapeutic agent dispersed within a polymer matrix, the polymer matrix comprising hyaluronic acid, or a derivative thereof, and a divalent metal cation.

16. The composition according to claim 15, wherein the therapeutic agent is a protein or peptide.

17. A method for the production of microparticles suitable for pulmonary administration, comprising the steps of:
(a) mixing a therapeutic active with a hyaluronic acid polymer to form an aqueous gel,
(b) adding the gel to a stirred non-aqueous solvent to form a dispersion of gel microdroplers, and
(c) removing the non-aqueous solvent to form dried microparticles.

18. The method according to claim 17, wherein the solvent is removed by spray-drying.

19. The method according to claim 17, wherein the solvent is a perfluorocarbon.

20. The method according to claim 17, wherein the solvent is perfluorodecalin or perfluoro-n-octane.

21. A method for the production of microparticles suitable for pulmonary administration, comprising mixing divalent metal cations with an aqueous solution comprising a therapeutic agent and a hyaluronic acid, or a salt thereof, to form a solution or suspension and processing the resulting product to form microparticles.

22. The method according to claim 21, wherein the divalent metal cations are selected from the group consisting of zinc, lithium, calcium, ammonium, magnesium, copper and cobalt.

23. The method according to claim 17, wherein the therapeutic agent is a protein or peptide.

24. The method according to claim 23, wherein the agent is insulin.

25. The method according to claim 17, wherein the microparticles are form 0.1 to 40 μm in diameter.

26. The method according to claim 25, wherein the microparticles are form 0.1 to 5 μm in diameter.

27. The composition, according to claim 16, wherein the agent is insulin.

28. The method, according to claim 21, wherein the therapeutic agent is a protein or peptide.

29. The method, according to claim 28, wherein the agent is insulin.

30. The method, according to claim 21, wherein the microparticles are form 0.1 to 40 μm in diameter.

31. The method, according to claim 30, wherein the microparticles are form 0.1 to 5 μm in diameter.

32. A method for delivering a therapeutic agent to a patient in need of therapy, wherein said method comprises administering to the patient a microparticle comprising a therapeutic agent dispersed within a polymer matrix, the polymer matrix comprising a first polymer of hyaluronic acid, or a derivative thereof, and a second biodegradable polymer.

33. The method, according to claim 32, wherein the second polymer is a non-ionic polymer, a polymeric gum, or a combination thereof.

34. The method, according to claim 32, wherein the therapeutic agent is a protein or peptide, the second polymer is a water-soluble polysaccharide containing two or more different sugar sub-units and the microparticle has a diameter less than 30 μm.

35. The method, according to claim 32, wherein the therapeutic agent is a protein or peptide, the second polymer is a homopolymeric gum and the microparticle has a diameter less than 30 μm.

36. The method, according to claim 32, wherein the second biodegradable polymer is cellulose or a cellulose derivative.

37. The method, according to claim 36, wherein the second polymer is carboxymethyl cellulose, hydroxypropylmethyl cellulose, hydroxyethyl cellulose, hydroxypropyl cellulose, or a mixture thereof.

38. The method, according to claim 32, further comprising a cross-linking agent.

39. The method, according to claim 32, wherein the therapeutic agent is a protein or peptide.

40. The method, according to claim 39, wherein the agent is insulin.

41. The method, according to claim 32, wherein the microparticle has a size from 0.1 to 40 μm in diameter.
